(19) United States
(12) Patent Application Publication
(10) Pub. No.: US 2025/0241759 A1
(43) Pub. Date: Jul. 31, 2025

(54) IMPLANT SECURING SYSTEMS FOR TISSUE REPAIR AND METHOD THEREOF

(71) Applicant: ANIKA THERAPEUTICS, INC., Bedford, MA (US)

(72) Inventor: Daniel Norton, Fort Wayne, IN (US)

(21) Appl. No.: 19/038,535

(22) Filed: Jan. 27, 2025

Related U.S. Application Data
(60) Provisional application No. 63/625,276, filed on Jan. 25, 2024.

(51) Int. Cl. A61F 2/30; A61F 2/32; A61F 2/38; A61F 2/40; A61F 2/42; A61L 27/20; A61L 27/38; A61L 27/54

(52) U.S. Cl. CPC A61F 2/30756; A61F 2/30749; A61F 2/32; A61F 2/38; A61F 2/40; A61F 2/4202; A61L 27/20; A61L 27/3852; A61L 27/54; A61F 2002/30751; A61F 2002/30766

(57) ABSTRACT

Embodiments described herein relate to a patch securing system including a patch implant, and a suture system configured to translocate the patch implant into a patient and secure it to a desired tissue. In some embodiments, a system is configured to dispose an implant in a patient and to secure the implant to a desired tissue includes a patch, a sleeve having a lumen extending through the longitudinal axis of the sleeve, where the sleeve is configured to reversibly transition between a constricted configuration and an expanded configuration, a first constricting suture configured to pass at least partially through the lumen and the patch, and a second constricting suture configured to pass at least partially through the lumen and the patch, where the first constricting suture and the second constricting suture are configured to translocate the implant to a location contacting the desired tissue and to secure the implant thereto.

### CROSS-REFERENCE TO RELATES APPLICATIONS

This application claims the benefit of U.S. Provisional Patent Application No. 63/625,276, filed on Jan. 25, 2024, which is fully incorporated herein by reference.

### BACKGROUND

Implants including tissue engineered scaffolds in the form of patches can be difficult to place and secure to a desired tissue due to tearing of the scaffold and a lack of proper attachment means to secure and maintain the scaffolds to the desired tissue location for healing and regeneration. Traditional methods have included invasive surgical procedures which increase operating time and risk of infection for a patient. Traditional methods of securing tissue scaffolds to patients includes suturing around the outside of the patch which can be time consuming and also increases operating time and risk of infection to the patient.

### BRIEF SUMMARY

In one aspect, systems, methods and devices described herein relate to disposing an implant in a patient and securing the implant to a desired tissue. In some embodiments, the system includes a patch, a sleeve having a lumen extending through the longitudinal axis of the sleeve, wherein the sleeve is configured to reversibly transition between a constricted configuration and an expanded configuration, a first constricting suture configured to pass at least partially through the lumen and the patch, and a second constricting suture configured to pass at least partially through the lumen and the patch, wherein the first constricting suture and the second constricting suture are configured to translocate the implant to a location contacting the desired tissue and to secure the implant thereto.

In another aspect, systems, methods and devices described herein relate to a system to secure an implant to a desired tissue in a patient includes a sleeve having a lumen extending along a longitudinal axis of the sleeve, where the sleeve is configured to reversibly transition between a constricted configuration and an expanded configuration, a first constricting suture configured to pass at least partially through the lumen and the implant, and a second constricting suture configured to pass at least partially through the lumen and the implant, where the first constricting suture and the second constricting suture are configured to translocate the implant to a location contacting the desired tissue and to secure the implant thereto, where the implant is in the form of a patch. The system may also include where the patch includes a textile material includes one or more threads and/or fibers. The system may also include where the patch includes a non-woven scaffold material. The system may also include where the patch includes at least one of hyaluronic acid, collagen, esterified hyaluronic acid, and any combination thereof. The system may also include where the patch is configured to be used to elucidate hyaline-like cartilage regeneration in at least one of a knee, an ankle, a shoulder, and a hip. The system may also include where the system further includes a first trap configured to receive the first constricting suture therethrough. The system may also include where the system further includes a deployment tool, configured to dispose the sleeve into the desired tissue when compressed, where the sleeve is detachably coupled at a distal end of the deployment tool. Other technical features may be readily apparent to one skilled in the art from the following figures, descriptions, and claims.

In another aspect, a method for placing and securing an implant at a location adjacent to a desired tissue includes placing the implant at the location adjacent to the desired tissue, transitioning a sleeve from an expanded configuration to a constricted configuration, the sleeve having a lumen extending along a longitudinal axis of the sleeve, where the sleeve is configured to reversibly transition between the constricted configuration and the expanded configuration, disposing a first constricting suture at least partially through the lumen of the sleeve and the implant, and disposing a second constricting suture at least partially through the lumen of the sleeve and the implant, where the first constricting suture and the second constricting suture are configured to translocate the implant to a location contacting the desired tissue thereby securing the implant to the desired tissue, where the implant is in the form of a patch. The method may also include where the implant includes a patch includes a textile material includes one or more threads and/or fibers. The method may also include where the patch includes a non-woven scaffold material. The method may also include where the patch includes at least one of hyaluronic acid, collagen, esterified hyaluronic acid, and any combination thereof. The method may also include where the patch is configured to be used to elucidate hyaline-like cartilage regeneration in at least one of a knee, an ankle, a shoulder, and a hip. The method may also include the method further includes forming a first trap configured to receive the first constricting suture therethrough. The method may also include the method further includes using a deployment tool, configured to dispose the sleeve into the desired tissue when compressed, where the sleeve is detachably coupled at a distal end of the deployment tool. Other technical features may be readily apparent to one skilled in the art from the following figures, descriptions, and claims. The system may also include where the system further includes a second trap configured to receive the second constricting suture therethrough. The system may also include where the system further includes a constricting suture portion extends between the first trap and the second trap to form a loop, where the constricting suture portion includes the first constricting suture and the second constricting suture. The system may also include where the constricting suture portion is configured to extend through the lumen of the sleeve. The system may also include where the first trap and the second trap are configured to prevent the first constricting suture and the second constricting suture from sliding backwards when the sleeve is in the constricted position. The system may also include where the deployment tool is configured to be self-punching. The method may also include the method further includes forming a second trap configured to receive the second constricting suture therethrough. The method may also include configuring the first trap and the second trap to prevent the first constricting suture and the second constricting suture from sliding backwards when the sleeve is in the constricted position. The method may also include the method further includes forming a constricting suture portion extending between the first trap and the second trap, where the constricting suture portion includes the first constricting suture and the second constricting suture. The method may also include the method further includes extending the constricting suture portion through the lumen of the sleeve. The method may also include where the deployment tool is self-punching. Other technical features may be readily apparent to one skilled in the art from the following figures, descriptions, and claims.

### BRIEF DESCRIPTION OF THE SEVERAL VIEWS OF THE DRAWINGS

FIG. 1 illustrates an example of a patch securing system, according to an embodiment described herein.

FIG. 2A-FIG. 2M illustrate an example of a first surgical method which may be used to deploy a patch into a patient and a patch securing system, tools, and implants thereof.

FIG. 3 illustrates another example of a patch securing system, according to an embodiment described herein.

FIGS. 4A-FIG. 4I illustrate an example of a second surgical method which may be used to deploy a patch into a patient and a patch securing system, tools, and implants thereof.

### DETAILED DESCRIPTION

Unless defined otherwise, all technical and scientific terms used herein have the same meaning as is commonly understood by one of skill in the art to which the claimed subject matter belongs. Generally, nomenclatures utilized in connection with, and techniques of rotator cuff surgery, tendon surgery, and tools and methods thereof described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, the term “about” means within +10% of the value it modifies. For example, “about 1” means “0.9 to 1.1”, “about 2%” means” 1.8% to 2.2% “, “about 2% to 3%” means” 1.8% to 3.3%”, and “about 3% to about 4%” means “2.7% to 4.4%.” Unless otherwise clear from the context, all numerical values provided herein are modified by the term “about.”

As used herein, singular forms “a,” “and,” and “the” include plural referents unless the context clearly indicates otherwise. Thus, e.g., reference to “a tendon” includes a single tendon or a plurality of tendons.

The terms “a” and “an” are defined as one or more unless this disclosure explicitly requires otherwise.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.”

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20-fold, etc., as well as 1.1, 1.2, 1.3, 1.4, 1.5-fold, etc., 2.1, 2.2, 2.3, 2.4, 2.5-fold, etc., and so forth.

The term “coupled” is defined as connected, although not necessarily directly, and not necessarily mechanically; two items that are “coupled” may be unitary with each other.

The term “substantially” is defined as largely but not necessarily wholly what is specified (and includes what is specified; e.g., substantially 90 degrees includes 90 degrees and substantially parallel includes parallel), as understood by a person of ordinary skill in the art. In any embodiment of the present apparatuses, kits, and methods, the term “substantially” may be substituted with “within [a percentage] of” what is specified, where the percentage includes 0.1, 1, 5, and/or 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, an apparatus, device or system that is configured in a certain way is configured in at least that way, but it can also be configured in other ways than those specifically described.

Any embodiment of any of the present apparatuses and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

In reading the present application, the practitioner of ordinary skill in the art will appreciate that the terms “substantial” and “substantially” are intended to convey having a detectable amount and more than a negligible amount or degree of the attribute modified by the terms; the term “flexible” is intended to convey having the ability to be actively deformed and restored to its spatial condition and configuration with a minimum of persistent distortion, at least over some number of cycles, whereas the term “elastic” conveys having the characteristic of being flexible as well as having a tendency to self-restore to its original condition or configuration and, potentially, to urge other components and/or features to return to an earlier configuration. The term “elongate” is intended to convey the sense of a three dimensional object where one of the three dimensions is substantially longer than the other two dimensions.

Described herein, in yet other aspects, are fasteners for coupling an implant to tissue (e.g., soft-tissue and/or bone), fabric-like implants, and assemblies with fasteners pre-loaded with implants. The present implants generally comprise at least one flexible, fibrous layer that is substantially planar in a flattened state.

In some embodiments, the implant is a substantially flexible patch. In some embodiments, the implant The medical device can be tubes, membranes, non-woven fabrics, gauzes, sponges, and/or sutures. In some embodiments, the implant is a tissue scaffold. In some embodiments, the present implants generally comprise at least one flexible, fibrous layer that is substantially planar in a flattened state (e.g., a patch). In some of the present embodiments, the implant is a patch comprising a woven layer. In some embodiments, the patch comprises a woven layer and a nonwoven layer coupled to the woven layer (e.g., via stitches). In some configurations of the present implants, the implant further comprises at least one suture leg (e.g., two suture legs each) coupled to the implant at a point that is closer to the second end edge than to the first end edge, the suture leg having a free portion that is configured to extend beyond the second end edge.

In some embodiments, the implant is a flexible patch and has a first end edge, a second end edge, and a pair of lateral edges extending between the first and second end edges, where the implant is substantially planar when in a flattened state. For example, the patch may be flattened such that the first end edge is separated from the second end edge under a first tension of 5 Newtons (N) applied perpendicular to each of the end edges, and in which the lateral edges are separated from each other under a tension of 5 N applied perpendicular to each of the lateral edges.

As used herein a self-punching suture tool is a specialized medical device designed to aid in tissue repair during surgeries. Instead of requiring a separate punching instrument to create holes for sutures, the self-punching suture tool combines both functions, thereby streamlining the procedure. In some embodiments, the self punching tool has a perforative tip configured to create precise holes in the tissue, reducing the need for additional steps. In some embodiments, the self-punching tool comprises a self-locking mechanism to securely lock the sutures in place, thereby increasing reliability during procedures. In some embodiments, the self-punching tool is designed to minimizes tissue damage, which may help reduce recovery time.

Details associated with the embodiments described above and others are presented below.

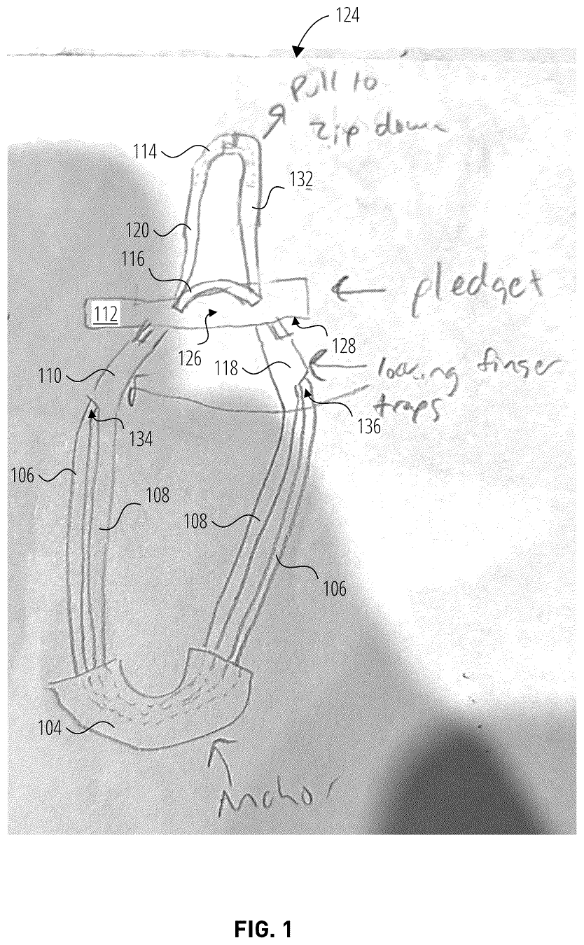

FIG. 1 illustrates an example of a patch securing system 124, according to an embodiment described herein. In some embodiments, the patch securing system 124 comprises a patch 112 and a suture system configured to secure the patch securing system 124 to a tissue in a human body.

In some embodiments, the patch 112 is a compliant material. In some embodiments, the patch 112 is a textile material comprising one or more threads and/or fibers. In some embodiments, patch 112 comprises a non-woven scaffold material. In some embodiments, the patch 112 is configured to promote cartilage regeneration. In some embodiments, the patch 112 is configured to be used in combination with mesenchymal stem cells (MSCs) to support hyaline-like cartilage regeneration in the knee, the ankle, the shoulder, and/or the hip. In some embodiments, the patch 112 comprises at least one of hyaluronic acid, collagen, esterified hyaluronic acid, or any combination thereof. In some embodiments, the patch 112 is Hyalofast™. In some embodiments, the patch 112 has a patch first surface 126 and a patch second surface 128.

In some embodiments, the suture system comprises a constricting suture first portion 114. In some embodiments, the constricting suture first portion 114 forms a loop having a constricting suture first portion first end 120 and a constricting suture first portion second end 132. In some embodiments, the constricting suture first portion first end 120 passes through a first area of the patch 112 and the constricting suture first portion second end 132 passes through a second portion of the patch 112 spaced apart from the first portion. In some embodiments, the constricting suture first portion first end 120 extends through the patch first surface 126 and the patch second surface 128 in a distal direction forming a deployment suture portion 106. In some embodiments, at least a portion of the deployment suture portion 106 passes through a sleeve 104. In some embodiments, the deployment suture portion 106 forms a loop after passing through the sleeve 104 where the end passing through the sleeve 104 extends in a direction towards the patch 112. In some embodiments, the end of the deployment suture portion 106, after passing through the sleeve 104, passes through a portion of the patch 112. In some embodiments, the deployment suture portion 106 forms a constricting suture second portion 116. In some embodiments, the constricting suture second portion 116 passes through the patch 112 in two locations. In some embodiments, the two locations are the same locations formed by the constricting suture first portion first end 120 and the constricting suture first portion second end 132. In some embodiments, the constricting suture second portion 116 forms a loop. In some embodiments, the constricting suture second portion 116 extends through the patch 112 and forms a first trap 110.

In some embodiments, the first trap 110 comprises a first opening 134 extending through the longitudinal axis of the first trap 110. In some embodiments, the first opening 134 is configured to receive a deployment suture portion 106 therethrough. In some embodiments, the first trap 110 is configured to have a protrusion extending from the outer surface of constricting suture portion 108 forming a flexible cylinder oriented along the longitudinal axis of the constricting suture portion 108.

In some embodiments, the constricting suture first portion second end 132 extends through the patch 112 and forms a second trap 118. In some embodiments, the second trap 118 comprises a second opening 136 extending through the longitudinal axis of the second trap 118. In some embodiments, the second opening 136 is configured to receive a deployment suture portion 106 therethrough. In some embodiments, the second trap 118 is configured to have a protrusion extending from the outer surface of constricting suture portion 108 forming a flexible cylinder oriented along the longitudinal axis of the constricting suture portion 108.

In some embodiments, a constricting suture portion 108 extends from the first trap 110 and the second trap 118 to form a loop. In some embodiments, the constricting suture portion 108 is located at a position distal to the patch 112. In some embodiments, the constricting suture portion 108 extends through the lumen of the sleeve 104.

In some embodiments, the patch securing system 124 comprises the sleeve 104. In some embodiments, the sleeve 104 is configured to transition between a constricted configuration and an expanded configuration. In some embodiments, the sleeve 104 is configured to secure the patch 112 to a tissue in a patient. In some embodiments, the patch 112 is disposed adjacent to a tissue in the human body and the sleeve 104 in the constricted configuration applies tension to the tissue to secure the patch 112 to the tissue. In some embodiments, the sleeve 104 has a center lumen extending through the longitudinal axis thereof. The center lumen of the sleeve 104 may be configured to receive one or more sutures. For example, the sleeve 104 may be configured to receive a deployment suture portion 106 and a constricting suture portion 108. In some embodiments, the sleeve 104 is a soft tissue anchor.

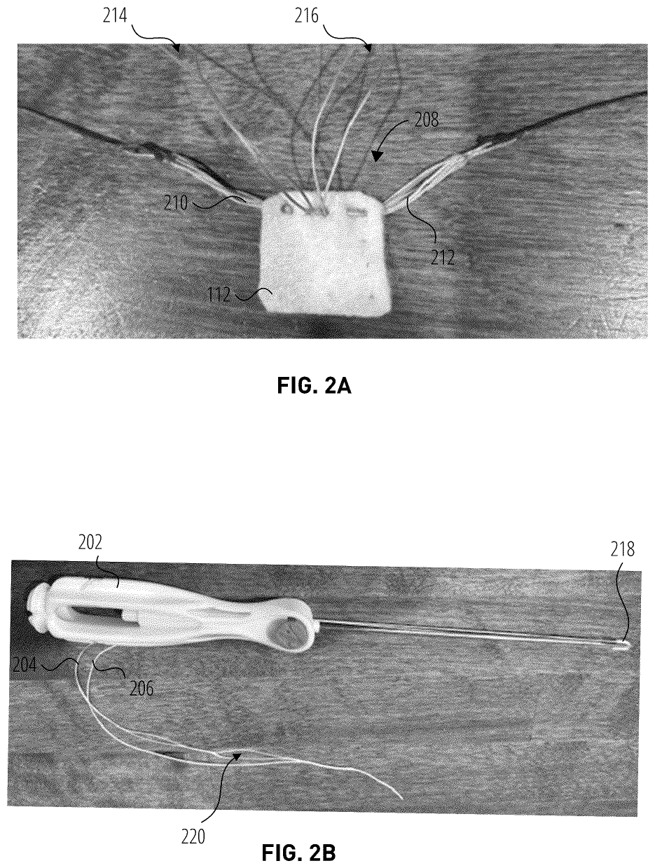

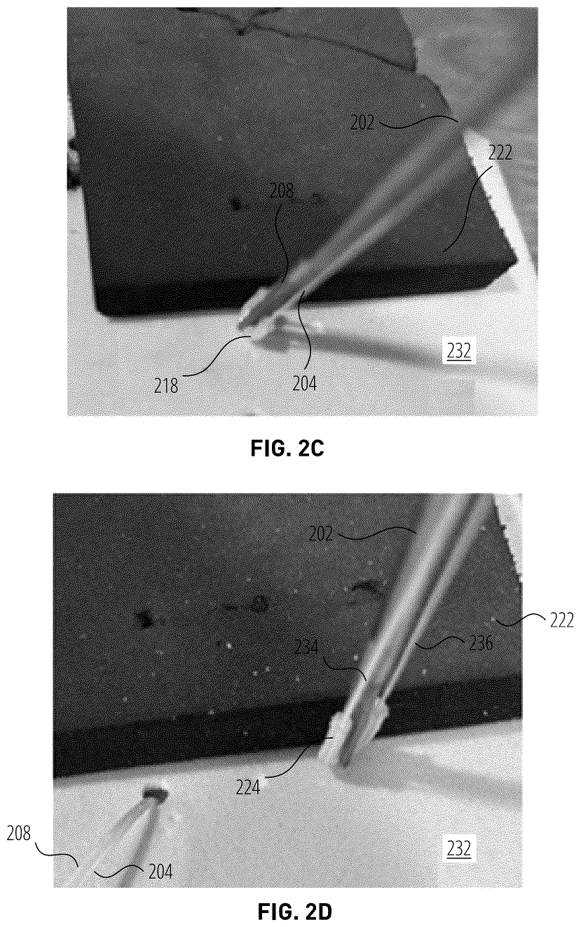

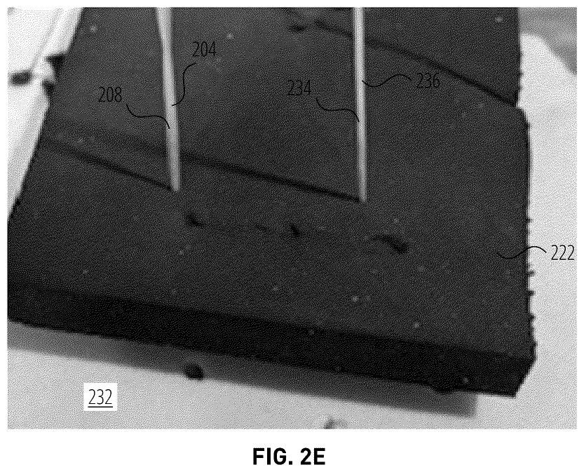

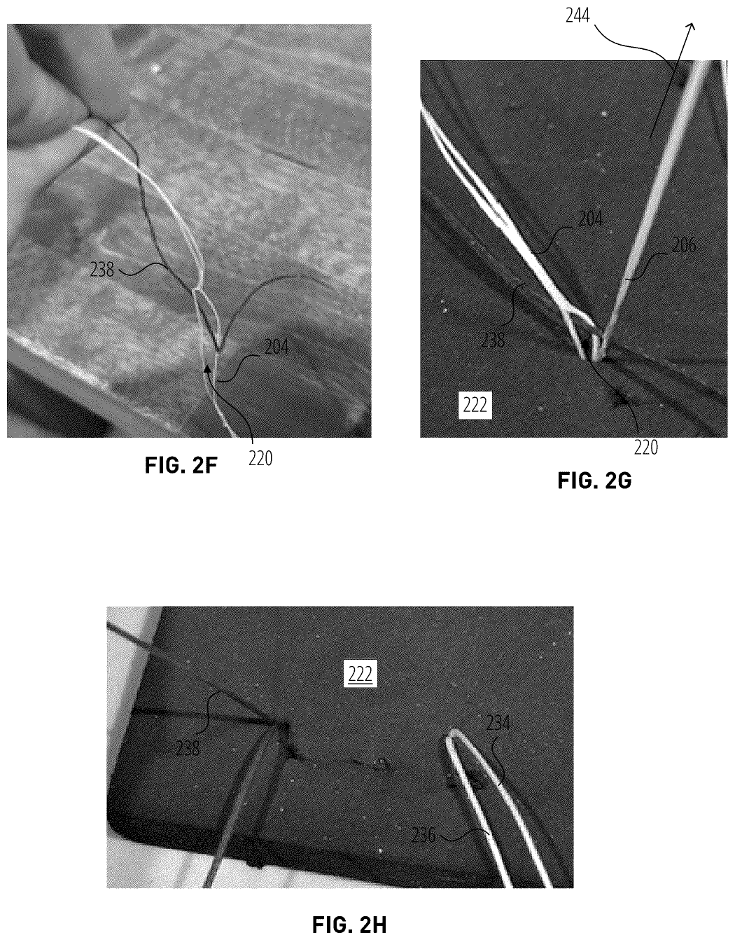

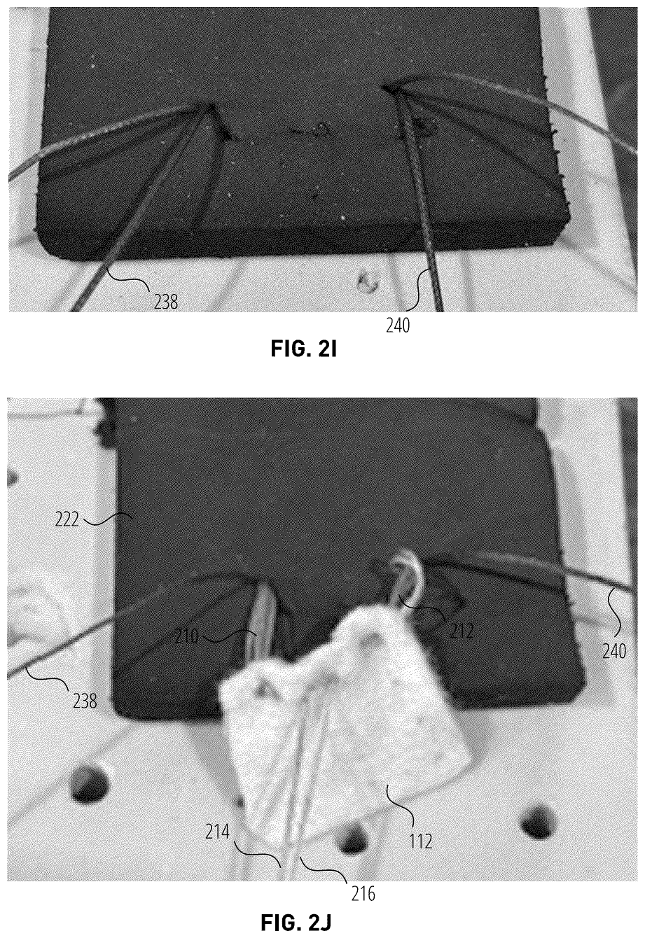

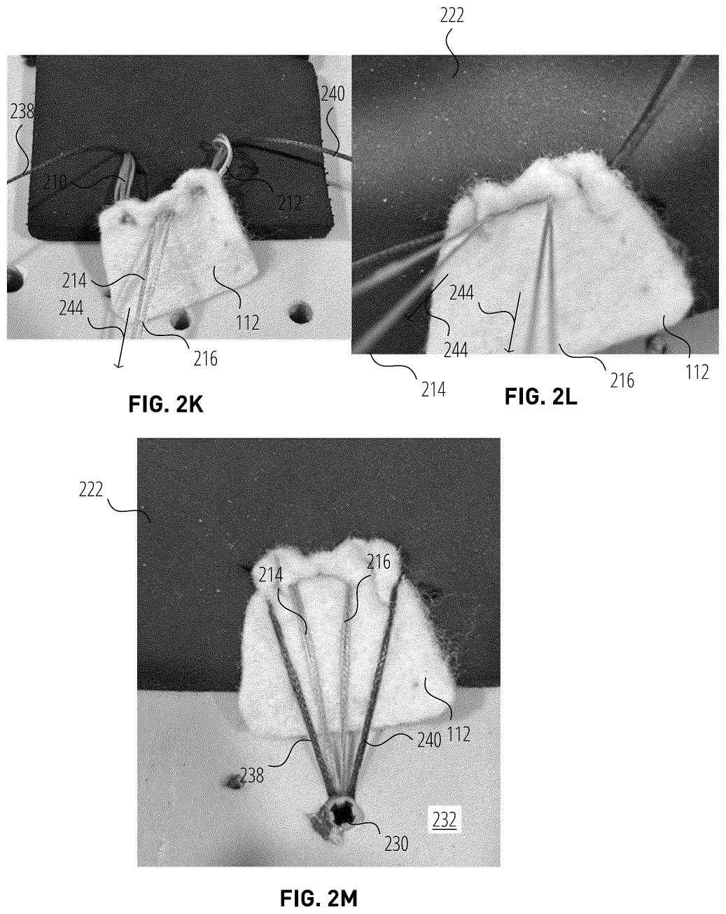

FIG. 2A-FIG. 2M illustrate an example of a first surgical method which may be used to deploy a patch 112 into a patient and a patch securing system 208, tools, and implants thereof.

In some embodiments, the method includes disposing one or more sutures through a patch 112 for example, as shown in FIG. 2A. A first adjustable loop 210 is disposed through a first corner of the patch 112 and a second adjustable loop 212 is disposed through a second corner adjacent to the first corner. In some embodiments, a first zip strand 214 is disposed through the patch 112. In some embodiments, a second zip strand 216 is disposed through the patch 112. In some embodiments, the first zip strand 214 and the second zip strand 216 are disposed through a portion of the patch 112. In some embodiments, the first zip strand 214 and the second zip strand 216 are disposed through the patch 112 at a location spaced apart from and in line with the location where the first adjustable loop 210 and the second adjustable loop 212 pass through the patch 112. In some embodiments, the first adjustable loop 210, the first zip strand 214, the second zip strand 216, and the second adjustable loop 212 are coupled together. In some embodiments, the patch securing system 208 is configured such that pulling on one or both of the first zip strand 214 and the second zip strand 216 causes one or more of the first adjustable loop 210 and the second adjustable loop 212 to contract. In some embodiments, contracting the first adjustable loop 210 and/or the second adjustable loop 212 causes the patch 112 to move in a distal direction towards the desired tissue location. In some embodiments, once the patch 112 abuts or is disposed adjacent to the desired tissue location, further tension on the first zip strand 214 and the second zip strand 216 will cause the first adjustable loop 210 and the second adjustable loop 212 to contract from into a contracted configuration and thereby couple the patch 112 to the desired tissue. In some embodiments, the contracted configuration is a knot.

In some embodiments, a deployment tool 202 (see FIG. 2B) is configured to deploy the patch securing system 208 into a desired tissue. For example, the deployment tool 202 may be configured to detachably couple a first soft tissue anchor 218 at a distal end of the deployment tool 202. The first soft tissue anchor 218 may be the sleeve 104 as shown and described in reference to FIG. 1. In some embodiments, the first soft tissue anchor 218 has a center lumen configured to receive a first deployment suture 206 and a second deployment suture 204. In some embodiments, the first deployment suture 206 and the second deployment suture 204 are coupled with and pass through a patch 112. In some embodiments, at least one of the second deployment suture 204 and the first deployment suture 206 comprise a bifurcation portion 220.

In some embodiments, the method includes disposing a first soft tissue anchor 218 into a first tissue 232 with the deployment tool 202 as shown in FIG. 2C. For example, the first soft tissue anchor 218 may be disposed into the first tissue 232 at a location adjacent to the desired tissue 222. In some embodiments, the desired tissue 222 is a rotator cuff. In some embodiments, the first soft tissue anchor 218 is disposed into the first tissue 232 by applying pressure to the distal portion of the deployment tool 202, thereby applying compressive force along the longitudinal axis of the deployment tool 202. In some embodiments, the deployment tool 202 is self-punching.

In some embodiments, the method includes disposing a second soft tissue anchor 224 into the first tissue 232 as shown in FIG. 2D. In some embodiments, the second soft tissue anchor 224 is disposed into the first tissue 232 at location in line with the first soft tissue anchor 218 and adjacent to the desired tissue 222. In some embodiments, a first deployment suture of the second suture anchor 234 and a second deployment suture of the second suture anchor 236 extend from a center lumen extending through the second soft tissue anchor 224.

In some embodiments, a suture passer tool (not shown) is used to pass the patch securing system 208, the second deployment suture 204, the first deployment suture of the second suture anchor 234, and the second deployment suture of the second suture anchor 236 through the desired tissue 222 as shown in FIG. 2E.

In some embodiments, the method includes passing a first medial suture 238 through a bifurcation portion 220 of at least one of patch securing system 208, the second deployment suture 204, the first deployment suture of the second suture anchor 234, and the second deployment suture of the second suture anchor 236 as shown in FIG. 2F. In some embodiments, the method includes pulling on the first deployment suture 206 in a pull direction 244 so as to move the bifurcation portion 220 and the first medial suture 238 through the desired tissue 222 to a location abutting or patch adjacent to the 218 as shown in FIG. 2G. In some embodiments, the method includes repeating the steps described above to move a second medial suture 240 to a location adjacent to or abutting second soft tissue anchor 224 through using a bifurcation region in the first deployment suture of the second suture anchor 234 or the second deployment suture of the second suture anchor 236 as shown in FIG. 2H and FIG. 2I.

In some embodiments, the method includes coupling the first medial suture 238 with the first adjustable loop 210. In some embodiments, the method includes coupling the second medial suture 240 with the second adjustable loop 212 as shown in FIG. 2J. In some embodiments, the method includes passing at least a portion of the first adjustable loop 210 and/or the second adjustable loop 212 through the patch 112. In some embodiments, the method includes coupling the second adjustable loop 212 with the first zip strand 214, the second zip strand 216, and the first adjustable loop 210. In some embodiments, the method includes pulling on at least one of the first medial suture 238 and the second medial suture 240 in a direction away from the desired tissue 222 so as to contract and/or move the first adjustable loop 210 and the second adjustable loop 212 to the first soft tissue anchor 218 and the second soft tissue anchor 224 and therefore move the patch 112 from a first location to the desired tissue 222 to a second position closer to the desired tissue 222 than the first location as shown in FIG. 2J-FIG. 2L. FIG. 2L illustrate an example of the patch 112 located at a location in contact with the desired tissue 222. In some embodiments, once the first medial suture 238 and second medial suture 240 are fully through the first soft tissue anchor 218 and the second soft tissue anchor 224 respectively they are unable to slide back in the opposite direction. For example, the first medial suture 238 and the second medial suture 240 may be unable to slide back because the first adjustable loop 210 and/or the second adjustable loop 212 are disposed within the first soft tissue anchor 218 and the second soft tissue anchor 224.

In some embodiments, the method includes pulling the first zip strand 214 and the second zip strand 216 in a pull direction 244 as shown in FIG. 2K and FIG. 2L. Pulling on the first zip strand 214 and the 216 may cause the loops to tighten and therefore attach the patch 112 to the desired tissue 222.

In some embodiments, the method includes securing the first zip strand 214, the second zip strand 216, the first medial suture 238, and the second medial suture 240 to a third tissue anchor 230 as shown in FIG. 2M. In some embodiments, the third tissue anchor 230 is located in a region lateral to the first soft tissue anchor 218 and the second soft tissue anchor 224. In some embodiments, the third tissue anchor 230 is disposed in the desired tissue 222. In some embodiments, the third tissue anchor 230 is disposed in the first tissue 232. In some embodiments, the third tissue anchor 230 is an X-Twist™ anchor. In some embodiments, the third tissue anchor 230 is a soft tissue anchor.

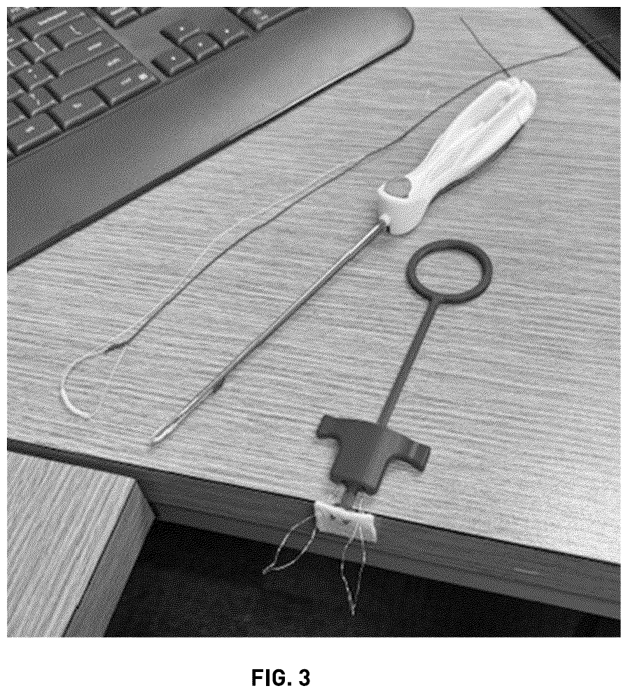

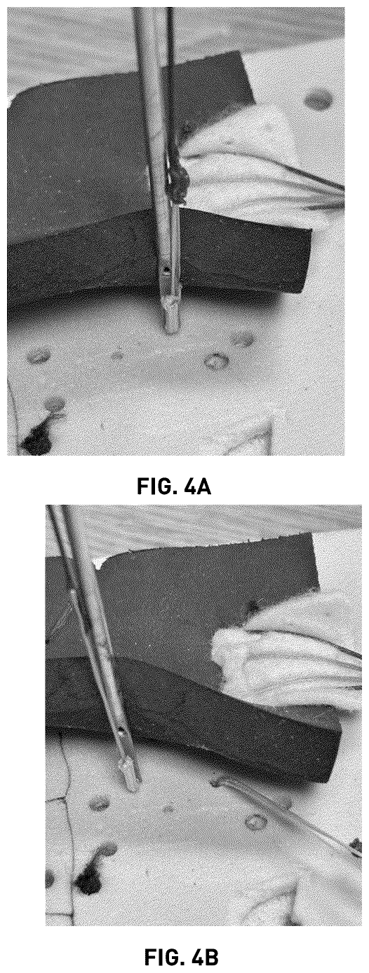

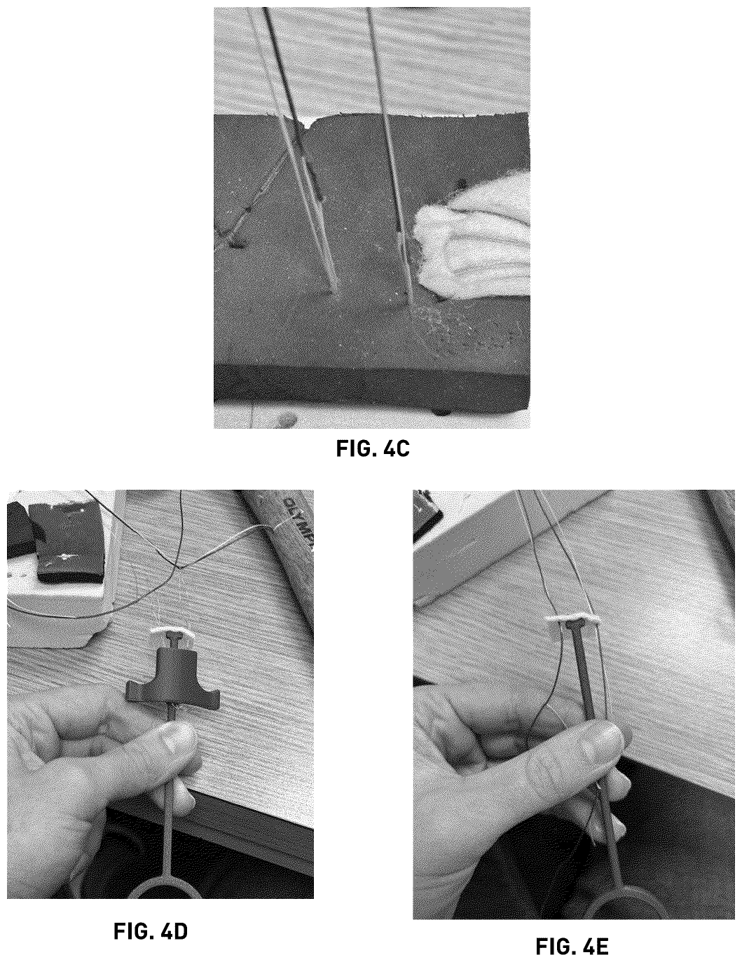

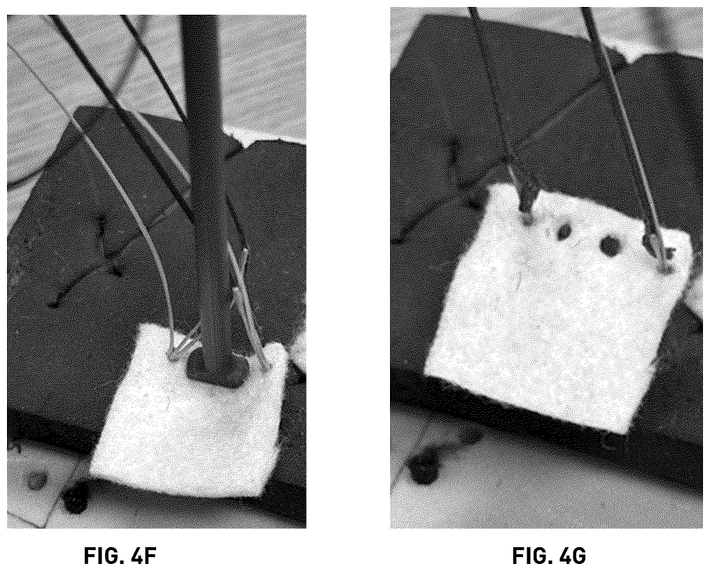

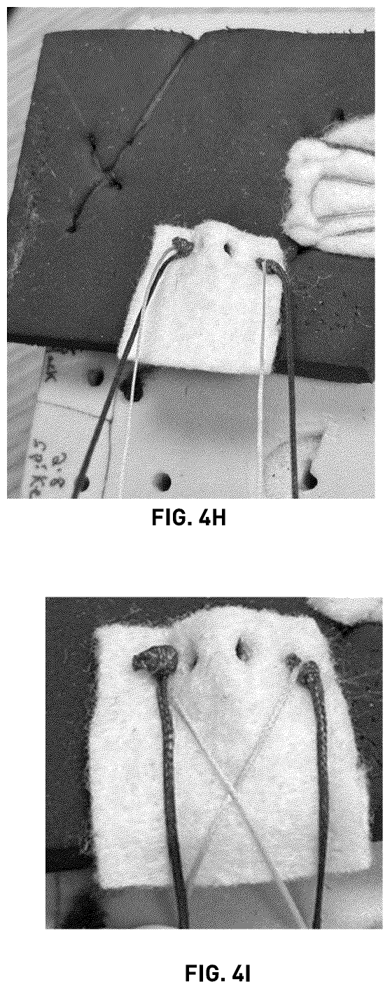

FIGS. 3-4I describe another exemplary embodiment for a patch securing system 300, and a method for its use. In some embodiments, the patch securing system 300 is used to secure a soft tissue, for example a tendon, to another tissue, such as bone (as described herein). For example, the system 300 may be used to secure a tendon at the rotator cuff (as described herein).

In some embodiments, the system 300 includes a sleeve 306, a patch 308, and a suture construct that includes a constricting suture 302 and a trapping suture 304. In some embodiments, the sleeve acts as a soft tissue anchor configured to be secured within a bore of another tissue, such as a bone (e.g., humerus). For example, the constricting suture 302 may be configured to pass through and within the sleeve 306, whereby pulling on one end of the constricting suture may cause the sleeve (after being inserted into a bore of a tissue) to transition to a deployed configuration that allows for the sleeve 306 to interact with the walls of the bore, and thereby enable securement therein. In some embodiments, the constricting suture 302 is secured to one end 307 of the sleeve (hereinafter referred to as “primary end”). For example, the constricting suture 302 at one end (hereinafter referred to as “secured end” of the constricting suture) may be bunched together, knotted together, or otherwise in a configuration that prevents or minimizes the ability for said secured end of the constricting suture 302 to pass through the sleeve 306.

In some embodiments, the remaining portion of the constricting suture 302 (e.g., that extends from said secured end) passes through the sleeve 306 and through a trapping site of the trapping suture 304. In some embodiments, the trapping site comprises a distal opening 310 at or about a first end of the trapping suture 304, a proximal opening 312 that is located proximal to the distal opening 310, and a passageway therethrough. In some embodiments, the trapping suture 304 includes a leading end that extends proximally from the proximal opening 312. In some embodiments, the constricting suture 302 is configured to enter through the distal opening 310 of the trapping suture 304, pass through the passageway, and exit through the proximal opening 312 of the trapping suture 304. In some embodiments, the constricting suture 302, after exiting the trapping suture through the proximal opening 312, is configured to enter through the sleeve 306 at the primary end 307, pass through the sleeve 306 once more, wherein a leading end of the constricting suture 302 exits the sleeve 306 (at the end opposite the primary end 307) and extends therefrom.

Accordingly, tensioning the leading end of the constricting suture 302 may result in a) the sleeve 306 to be transitioned to the deployed configuration where it will be configured to interface with the inner walls of a bore within a bone or other tissue for securement therein; and/or b) the trapping suture 304 to be pulled at the proximal opening 312 of the trapping site distally so as to reconfigure the trapping suture 304, at least at the trapping site, to be partially folded, where two parts of the trapping site may be disposed at least partially side-by side.

In some embodiments, the system 300 includes two or more suture constructs, wherein a portion of each suture construct is configured to pass through a respective opening 320 (see FIG. 4C) through the patch 308. In some embodiments, the patch 308 includes any patch as described herein, including for example, patch 112 from FIGS. 1 and 2A.

In some embodiments, the system 300 further includes a deployment tool 314 for inserting the sleeve and suture construct at least partially within a bore of a tissue, such as bone.

FIGS. 4A-4I depict an exemplary method of securing a soft tissue, for example to another tissue, such as a bone, using the patch securing system 300. For example, the system 300 may be used to repair a torn tendon at the rotator cuff.

First, the sleeve 306 is inserted into a bore of a tissue, which may be via a deployment tool 314 (FIG. 4A). In some embodiments, the sleeve 306 is configured to act as a medial anchor for a rotator cuff tendon repair. In some embodiments, the suture construct is coupled to and within the sleeve, as described herein in FIG. 3. FIG. 4B depicts a second sleeve, coupled with a second suture construct, being inserted within a second bore of a tissue (e.g., bone, as described herein). In some embodiments, tensioning on any one of the constricting suture and the trapping suture may transition the respective sleeve to a deployed configuration, so as to enable securement within the respective bore.

Once both suture constructs have been inserted and/or secured within a respective bore of a tissue, the leading ends of the constricting suture 302 and trapping suture 304 for each suture construct is passed through a respective opening 320 through a soft tissue (e.g., rotator cuff tendon) 318 (FIG. 4C).

The suture constructs are then passed through respective openings on the patch 308 (FIG. 4E). In some embodiments, a respective passing loop 316 (FIG. 4D) is passed through each opening of the patch 308, wherein the constricting suture 302 and trapping suture 304 of each suture construct is passed through a loop of said passing loop 316, after which the passing loop is pulled through the respective opening of the patch, so as to pull the constricting suture 302 and the trapping suture 304 of the respective suture construct through said respective opening on the patch (see FIG. 4E).

The patch 308 may then be pushed down to the tissue 318 (FIGS. 4F and 4G). In FIG. 4G, a support tool 322 for the patch is removed.

The leading portion of the constricting suture 302 is then tensioned (e.g., pulled) so as to secure the patch to the tissue (FIG. 4H). As described herein, pulling on the constricting suture 302 may allow for said constricting suture exiting the proximal opening 312 of the trapping site (on the trapping suture) to pull and fold a portion of the trapping suture 304 at the trapping site, such that trapping site, in the folded configuration 324, is unable to pass through the respective opening on the patch 308.

Once the patch is secured, the leading ends of the constricting suture and the trapping suture may be secured laterally (FIG. 4I) using any suture securing mechanism known in the art (for example, using a knotless suture anchor, a sleeve as described herein, etc.).

### SELECTED EMBODIMENTS

Embodiment 1: A system to secure an implant to a desired tissue in a patient comprising: a sleeve having a lumen extending along a longitudinal axis of the sleeve, wherein the sleeve is configured to reversibly transition between a constricted configuration and an expanded configuration; a first constricting suture configured to pass at least partially through the lumen and the implant; and a second constricting suture configured to pass at least partially through the lumen and the implant, wherein the first constricting suture and the second constricting suture are configured to translocate the implant to a location contacting the desired tissue and to secure the implant thereto; wherein the implant is in the form of a patch.

Embodiment 2: The system of embodiment 1, wherein the patch comprises a textile material comprising one or more threads and/or fibers.

Embodiment 3: The system of embodiment 1, wherein the patch comprises a non-woven scaffold material.

Embodiment 4: The system of any one of embodiments 1 to 3, wherein the patch comprises at least one of hyaluronic acid, collagen, esterified hyaluronic acid, and any combination thereof.

Embodiment 5: The system of any one of embodiments 1 to 4, wherein the patch is configured to be used to elucidate hyaline-like cartilage regeneration in at least one of a knee, an ankle, a shoulder, and a hip.

Embodiment 6: The system of any one of embodiments 1 to 5, wherein the system further comprises a first trap configured to receive the first constricting suture therethrough.

Embodiment 7: The system of embodiment 6, wherein the system further comprises a second trap configured to receive the second constricting suture therethrough.

Embodiment 8: The system of embodiment 7, wherein the system further comprises a constricting suture portion extends between the first trap and the second trap to form a loop, wherein the constricting suture portion comprises the first constricting suture and the second constricting suture.

Embodiment 9: The system of embodiment 8, wherein the constricting suture portion is configured to extend through the lumen of the sleeve.

Embodiment 10: The system of embodiment 8 or 9, wherein the first trap and the second trap are configured to prevent the first constricting suture and the second constricting suture from sliding backwards when the sleeve is in the constricted position.

Embodiment 11: The system of any one of embodiments 1 to 10, wherein the system further comprises a deployment tool, configured to dispose the sleeve into the desired tissue when compressed, wherein the sleeve is detachably coupled at a distal end of the deployment tool.

Embodiment 12: The system of embodiment 11, wherein the deployment tool is configured to be self-punching.

Embodiment 13: A method for placing and securing an implant at a location adjacent to a desired tissue comprising: placing the implant at the location adjacent to the desired tissue; transitioning a sleeve from an expanded configuration to a constricted configuration, the sleeve having a lumen extending along a longitudinal axis of the sleeve, wherein the sleeve is configured to reversibly transition between the constricted configuration and the expanded configuration; disposing a first constricting suture at least partially through the lumen of the sleeve and the implant; and disposing a second constricting suture at least partially through the lumen of the sleeve and the implant, wherein the first constricting suture and the second constricting suture are configured to translocate the implant to a location contacting the desired tissue thereby securing the implant to the desired tissue; wherein the implant is in the form of a patch.

Embodiment 14: The method of embodiment 13, wherein the implant comprises a patch comprising a textile material comprising one or more threads and/or fibers.

Embodiment 15: The method of embodiment 13, wherein the patch comprises a non-woven scaffold material.

Embodiment 16: The method of any one of embodiments 13 to 15, wherein the patch comprises at least one of hyaluronic acid, collagen, esterified hyaluronic acid, and any combination thereof.

Embodiment 17: The method of any one of embodiments 13 to 16, wherein the patch is configured to be used to elucidate hyaline-like cartilage regeneration in at least one of a knee, an ankle, a shoulder, and a hip.

Embodiment 18: The method of any one of embodiments 13 to 17, the method further comprising forming a first trap configured to receive the first constricting suture therethrough.

Embodiment 19: The method of embodiment 18, the method further comprising forming a second trap configured to receive the second constricting suture therethrough.

Embodiment 20: The method of embodiment 19, configuring the first trap and the second trap to prevent the first constricting suture and the second constricting suture from sliding backwards when the sleeve is in the constricted position.

Embodiment 21: The method of embodiment 19, the method further comprising forming a constricting suture portion extending between the first trap and the second trap, wherein the constricting suture portion comprises the first constricting suture and the second constricting suture.

Embodiment 22: The method of embodiment 21, the method further comprising extending the constricting suture portion through the lumen of the sleeve.

Embodiment 23: The method of any one of embodiments 13 to 22, the method further comprising using a deployment tool, configured to dispose the sleeve into the desired tissue when compressed, wherein the sleeve is detachably coupled at a distal end of the deployment tool.

Embodiment 24: The method of embodiment 23, wherein the deployment tool is self-punching.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

All publications, patents, patent applications and other documents cited in this application are hereby incorporated by reference herein in their entireties for all purposes to the same extent as if each individual publication, patent, patent application or other document were individually indicated to be incorporated by reference for all purposes.

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

Moreover, the terms “anterior,” and “posterior,” and “distal,” and “proximal” and so forth, are used merely as labels for positions relative to each other and are not intended to impose positional requirements on their objects.

What is claimed is:

1. A system to secure an implant to a desired tissue in a patient comprising:
a sleeve having a lumen extending along a longitudinal axis of the sleeve, wherein the sleeve is configured to reversibly transition between a constricted configuration and an expanded configuration;
a first constricting suture configured to pass at least partially through the lumen and the implant; and
a second constricting suture configured to pass at least partially through the lumen and the implant, wherein the first constricting suture and the second constricting suture are configured to translocate the implant to a location contacting the desired tissue and to secure the implant thereto; wherein the implant is in the form of a patch.

2. The system of claim 1, wherein the patch comprises a textile material comprising one or more threads and/or fibers.

3. The system of claim 1, wherein the patch comprises at least one of hyaluronic acid, collagen, esterified hyaluronic acid, and any combination thereof.

4. The system of claim 1, wherein the patch is configured to be used to elucidate hyaline-like cartilage regeneration in at least one of a knee, an ankle, a shoulder, and a hip.

5. The system of claim 1, wherein the system further comprises a first trap configured to receive the first constricting suture therethrough.

6. The system of claim 5, wherein the system further comprises a second trap configured to receive the second constricting suture therethrough.

7. The system of claim 6, wherein the system further comprises a constricting suture portion extends between the first trap and the second trap to form a loop, wherein the constricting suture portion comprises the first constricting suture and the second constricting suture.

8. The system of claim 7, wherein the constricting suture portion is configured to extend through the lumen of the sleeve.

9. The system of claim 7, wherein the first trap and the second trap are configured to prevent the first constricting suture and the second constricting suture from sliding backwards when the sleeve is in the constricted position.

10. The system of claim 1, wherein the system further comprises a deployment tool, configured to dispose the sleeve into the desired tissue when compressed, wherein the sleeve is detachably coupled at a distal end of the deployment tool.

11. A method for placing and securing an implant at a location adjacent to a desired tissue comprising:
placing the implant at the location adjacent to the desired tissue;
transitioning a sleeve from an expanded configuration to a constricted configuration, the sleeve having a lumen extending along a longitudinal axis of the sleeve, wherein the sleeve is configured to reversibly transition between the constricted configuration and the expanded configuration;
disposing a first constricting suture at least partially through the lumen of the sleeve and the implant; and
disposing a second constricting suture at least partially through the lumen of the sleeve and the implant, wherein the first constricting suture and the second constricting suture are configured to translocate the implant to a location contacting the desired tissue thereby securing the implant to the desired tissue;
wherein the implant is in the form of a patch.

12. The method of claim 11, wherein the implant comprises a patch comprising a textile material comprising one or more threads and/or fibers.

13. The method of claim 11, wherein the patch comprises at least one of hyaluronic acid, collagen, esterified hyaluronic acid, and any combination thereof.

14. The method of claim 11, wherein the patch is configured to be used to elucidate hyaline-like cartilage regeneration in at least one of a knee, an ankle, a shoulder, and a hip.

15. The method of claim 11, the method further comprising forming a first trap configured to receive the first constricting suture therethrough.

16. The method of claim 15, the method further comprising forming a second trap configured to receive the second constricting suture therethrough.

17. The method of claim 16, configuring the first trap and the second trap to prevent the first constricting suture and the second constricting suture from sliding backwards when the sleeve is in the constricted position.

18. The method of claim 16, the method further comprising forming a constricting suture portion extending between the first trap and the second trap, wherein the constricting suture portion comprises the first constricting suture and the second constricting suture.

19. The method of claim 18, the method further comprising extending the constricting suture portion through the lumen of the sleeve.

20. The method of claim 11, the method further comprising using a deployment tool, configured to dispose the sleeve into the desired tissue when compressed, where in the sleeve is detachably coupled at a distal end of the deployment tool.
